(19) United States
(12) Patent Application Publication
(10) Pub. No.: US 2020/0309770 A1
(43) Pub. Date: Oct. 1, 2020

(54) CHEMILUMINESCENCE IMMUNOASSAY KIT FOR ADIPONECTIN, AND PREPARATION METHOD AND USE THEREOF

(71) Applicant: SHENZHEN YHLO BIOTECH CO., LTD., SHENZHEN (CN)

(72) Inventors: Fuzhen XIA, SHENZHEN (CN); Chungen QIAN, SHENZHEN (CN); Gang WANG, SHENZHEN (CN); Liang ZHU, SHENZHEN (CN); Yuxi HE, SHENZHEN (CN)

(21) Appl. No.: 16/313,911

(22) PCT Filed: Jul. 6, 2016
(86) PCT No.: PCT/CN2016/088924

§ 371 Date: Dec. 28, 2018

(30) Foreign Application Priority Data
Jun. 30, 2016 (CN) 201610505400.7

(51) Int. Cl. G01N 33/532; G01N 33/543; G01N 33/74; G01N 21/76

(52) U.S. Cl. CPC G01N 33/532; G01N 21/76; G01N 33/74; G01N 33/54306

(57) ABSTRACT

Provided are a chemiluminescence immunoassay kit for adiponectin, and a preparation method and a use thereof. The chemiluminescence immunoassay kit for adiponectin comprises: a solid carrier coated with an adiponectin monoclonal antibody; and an adiponectin monoclonal antibody marked with a chemiluminescence marker.

### FIELD

The present disclosure relates to the field of in vitro detection, and more particularly relates to an adiponectin chemiluminescence immunoassay kit, a preparation method, and a use thereof.

### BACKGROUND

Adipose tissue is mainly composed of a large number of aggregated fat cells. Adiponectin (ADPN) is an endogenous biologically active polypeptide or protein secreted by adipocytes and is present in circulating plasma in humans at a concentration of 3 μg/ml to 30 μg/ml. Adiponectin is also known as Acrp30, apM1, AdipoQ, GBP28. Initially, adiponectin was found in human subcutaneous adipose tissue, plasma and murine fat cells. The adiponectin in the human body consists of 244 amino acids with a molecular weight of 30 KD. Adiponectin consists of a secretory signal sequence at an amino terminus (aa 1-18), a specific sequence (aa 19-41), a set of collagen repeat sequences consisting of 22 amino acids (aa 42-107), and a globular sequence (aa 108-244). The globular region is a key site for the biological activity of adiponectin, which is similar in structure to TNF-α. Adiponectin is highly homologous to collagen VIII, X and complement C1q. Monomers and trimers of adiponectin are biologically active forms or receptor affinity ligands, which can specifically bind to the G protein coupled receptor type I or type II adiponectin receptor on the liver cell membrane or skeletal muscle, thereby regulating fatty acid oxidation and glucose metabolism.

Adiponectin is an insulin sensitizing hormone (An Insulin-sensitizing Hormone), which can improve insulin resistance (Insulin resistance) and arteriosclerosis. Studies in humans have found that adiponectin levels can predict the development of type II diabetes and coronary heart diseases, and show potential for anti-diabetes, anti-atherosclerosis, and inflammation in clinical trials.

The conventional method of detecting adiponectin is mainly enzyme linked immunosorbent assay. However, limited by the characteristics of enzyme linked immunoassay, the conventional method of detecting adiponectin is in an open space during the detection process, which easily causes cross-contamination between various reagents. In addition, since the detection process is mostly manual, the amount of reagents or samples is not very accurate, the operation process is extremely cumbersome and complicated, and it is prone to operational errors and poor detection precision.

### SUMMARY

Accordingly, it is necessary to provide an adiponectin chemiluminescence immunoassay kit with a high detection precision, a preparation method, and a use thereof.

An adiponectin chemiluminescence immunoassay kit includes: a solid-phase carrier coated with an adiponectin monoclonal antibody, and an adiponectin monoclonal antibody labeled with a chemiluminescence marker.

A method of preparing the aforementioned adiponectin chemiluminescence immunoassay kit includes the steps of:

mixing an adiponectin monoclonal antibody and a solid-phase carrier and reacting fully to obtain a solid-phase carrier coated with the adiponectin monoclonal antibody; and

mixing a chemiluminescence marker and a adiponectin monoclonal antibody and reacting fully to obtain an adiponectin monoclonal antibody labeled with the chemiluminescence marker.

The adiponectin chemiluminescence immunoassay kit is capable of performing adiponectin detection using a fully automated chemiluminescence immunoassay analyzer as a detection tool. The experiment shows this adiponectin chemiluminescence immunoassay kit has a detection sensitivity of 0.01 ng/mL, which is at least 10 times more sensitive than a conventional method of detecting adiponectin. This adiponectin chemiluminescence immunoassay kit has a higher detection precision.

### BRIEF DESCRIPTION OF THE DRAWINGS

FIG. 1 is a standard curve diagram of adiponectin obtained in Example 3; and

FIG. 2 is a standard curve diagram of adiponectin obtained in Example 7.

### DETAILED DESCRIPTION OF THE INVENTION

The above objects, features and advantages of the present invention will become more apparent by describing in detail embodiments thereof with reference to the accompanying drawings. Numerous specific details are set forth in the description below in order to provide a thorough understanding of the invention. However, the present invention can be implemented in many other ways than those described herein, and those skilled in the art can make similar modifications without departing from the scope of the present invention, and thus the present invention is not limited by the specific embodiments disclosed below.

An adiponectin chemiluminescence immunoassay kit according to an embodiment includes a solid-phase carrier coated with an adiponectin monoclonal antibody and an adiponectin monoclonal antibody labeled with a chemiluminescence marker.

In the solid-phase carrier coated with the adiponectin monoclonal antibody, the adiponectin monoclonal antibody can be a human source, genetically engineered or animal source.

In the adiponectin monoclonal antibody labeled with the chemiluminescence marker, the adiponectin monoclonal antibody can be the human source, genetically engineered or animal source.

The adiponectin monoclonal antibodies used in the solid-phase carrier coated with the adiponectin monoclonal antibody and the adiponectin monoclonal antibody labeled with the chemiluminescence marker may be the same or different.

In an embodiment, in the solid-phase carrier coated with the adiponectin monoclonal antibody, the solid-phase carrier is a magnetic particle having a linking group for a protein chemical reaction on a surface thereof, a silica-based material having a surface for physical adsorption of proteins, or a magnetic particle subjected to polymer surface treatment.

When the solid-phase carrier is the magnetic particle having the linking group for the protein chemical reaction on the surface thereof or the magnetic particle subjected to polymer surface treatment, preferably, a mass ratio of the adiponectin monoclonal antibody to the magnetic particle ranges from 1:25˜35.

The magnetic particle having the linking group for the protein chemical reaction on the surface thereof can be a magnetic particle having a group selected from the group consisting of: an amino group, a carboxyl group, a tosyl group, and an oxiranyl group on the surface thereof.

The magnetic particle having the linking group for the protein chemical reaction on the surface thereof has a particle size of 0.05 μm to 3 μm.

Taking the solid-phase carrier as the magnetic particle having a carboxyl group on the surface thereof as an example, a preparation process of the solid-phase carrier coated with the adiponectin monoclonal antibody can be as follows: obtaining a suspension of carboxylated magnetic particles, resuspending with a MES (2-(N-morpholine)ethanesulfonic acid) buffer followed by magnetic separation to remove supernatant, and then adding an EDC (1-ethyl-3-(3-dimethylaminopropyl)-carbodiimide) aqueous solution to activate the surface carboxyl group of the carboxylated magnetic particles, and then adding the adiponectin monoclonal antibody and resuspending at room temperature for 2 h to 10 h, and removing supernatant by magnetic separation and then resuspending with a Tris buffer to obtain the carboxylated magnetic particles coated with the adiponectin monoclonal antibody. MES (2-(N-morpholine)ethanesulfonic acid) buffer has a concentration of 0.02 M and a pH of 5.5. Tris buffer has a concentration of 0.1 M and contains 2% of BSA, and has a pH of 8.0. EDC (1-ethyl-3-(3-dimethylaminopropyl)-carbodiimide) aqueous solution has a concentration of 10 mg/mL to 20 mg/mL, and a ratio of EDC to the carboxylated magnetic particles ranges from 0.05:0.1˜1.

In another embodiment, in the solid-phase carrier coated with the adiponectin monoclonal antibody, the solid-phase carrier is coupled with streptavidin, avidin or neutravidin, and the adiponectin monoclonal antibody is coupled with biotin.

The solid-phase carrier coated with the adiponectin monoclonal antibody may be a commercially available avidin solid-phase carrier, or may be an avidin solid-phase carrier prepared by avidin chemical bond and coupling to other solid-phase carriers.

In the adiponectin monoclonal antibody labeled with the chemiluminescence marker, the chemiluminescence marker is isluminol, terpyridine ruthenium, acridinium ester, alkaline phosphatase, or horseradish peroxidase.

Preferably, in a chemiluminescence marker labeled with a statin monoclonal antibody, a ratio of the adiponectin monoclonal antibody to the chemiluminescence marker ranges from 50:1˜10.

In an embodiment, in the adiponectin monoclonal antibody labeled with the chemiluminescence marker, the chemiluminescence marker is coupled with streptavidin, avidin or neutravidin, and the adiponectin monoclonal antibody is coupled with biotin.

Taking the chemiluminescence marker coupled with streptavidin and the adiponectin monoclonal antibody coupled with biotin as an example, and taking the chemiluminescence marker as acridinium ester as an example, a preparation process of the adiponectin monoclonal antibody labeled with the chemiluminescence marker is as follows: the adiponectin monoclonal antibody solution is taken, 500 μL of phosphate buffer with a pH of 8.0 is added thereto, and 0.1 mg of biotin succinimide (Biotin-NHS) is added and mixed, and is reacted at room temperature in the dark. After 1 h to 2 h, the reaction mixture is taken out and desalted by a centrifugal desalting column. During the desalting process, the treatment is performed firstly by purified water and TBS buffer, respectively, and the liquid in the desalting column is collected and stored to obtain the adiponectin monoclonal antibody solution labeled with the biotin. The streptavidin solution is taken, 0.1 M to 0.2 M of carbonate buffer with a pH of 9.0 to 9.5 is taken and mixed, and then the acridinium ester is added and mixed, and reacted at room temperature in the dark. After 1 h to 2 h, the reaction mixture is taken out and desalted by zeba centrifugal desalting column. During the desalting process, the treatment is performed firstly by purified water and TBS buffer, respectively, and finally the obtained streptavidin labeled with the acridinium ester is added. The adiponectin monoclonal antibody solution labeled with the biotin and the streptavidin labeled with the acridinium ester are mixed to obtain the adiponectin monoclonal antibody labeled with the acridinium ester.

In another embodiment, in the adiponectin monoclonal antibody labeled with the chemiluminescence marker, the chemiluminescence marker is directly linked to the adiponectin monoclonal antibody by a chemical reaction bond or is linked to the adiponectin monoclonal antibody by a protein crosslinking agent.

The protein crosslinking agent can be at least one of a carbonyldiimide salt and a succinimide.

Preferably, the protein crosslinking agent is at least one selected from the group consisting of 1-ethyl-(3-dimethylaminopropyl)carbonyldiimide, 1-ethyl-(3-dimethylaminopropyl)carbonyldiimide hydrochloride, N-hydroxysuccinimide, and sulfonated N-hydroxysuccinimide.

Taking the chemiluminescence marker as the acridinium ester as an example, a process in which the chemiluminescence marker is directly linked to the adiponectin monoclonal antibody by the chemical reaction bond to produce the adiponectin monoclonal antibody labeled with the chemiluminescence marker is as follows: the adiponectin monoclonal antibody solution is taken, a carbonate buffer is added and mixed, and then the acridinium ester is added and mixed, and reacted at room temperature in the dark. After 1 h to 2 h, the reaction mixture is taken out and desalted by the centrifugal desalting column. During the desalting process, the treatment is performed firstly by purified water and TBS buffer, respectively to obtain the adiponectin monoclonal antibody labeled with the acridinium ester.

Taking the chemiluminescence marker as alkaline phosphatase and the protein crosslinking agent as 1-ethyl-(3-dimethylaminopropyl)carbonyldiimide hydrochloride and N-hydroxysuccinimide as an example, a process in which the chemiluminescence marker is linked to the adiponectin monoclonal antibody by the protein crosslinking agent to produce the adiponectin monoclonal antibody labeled with the chemiluminescence marker is as follows: 1 mg of adiponectin monoclonal antibody solution and 1 mg of alkaline phosphatase are taken, 500 μL of 2-(N-morpholine)ethanesulfonic acid (MES) buffer with a pH of 5.5 is added, and 1 mg of 1-ethyl-(3-dimethylaminopropyl)carbodiimide hydrochloride and 1 mg of N-hydroxysuccinimide are added and mixed, and reacted at room temperature in the dark. After 1 h to 2 h, the reaction mixture is taken out and desalted by the centrifugal desalting column. During the desalting process, the treatment is performed firstly by purified water and TBS buffer, respectively, and the liquid in the desalting column is collected and stored to obtain the adiponectin monoclonal antibody solution labeled with the alkaline phosphatase.

In alternative embodiments, the aforementioned adiponectin chemiluminescence immunoassay kit further includes a chemiluminescence substrate solution.

The chemiluminescence substrate solution includes solution A and solution B. The solution A can be a H₂O₂ solution, and the solution B can be a NaOH solution.

In the present embodiment, the solution A is a H₂O₂ solution with a concentration of 0.1 mol/L, and the solution B is a NaOH solution with a concentration of 0.25 mol/L.

In alternative embodiments, the aforementioned adiponectin chemiluminescence immunoassay kit further includes an adiponectin calibrator.

A preparation process of the adiponectin calibrator is as follows: adiponectin is formulated into a solution of adiponectin at a concentration of 0 to 200 ng/mL using a calibrator buffer.

Specifically, the adiponectin calibrator is a solution of adiponectin at concentrations of 0 ng/mL, 0.5 ng/mL, 10 ng/mL, 20 ng/mL, 80 ng/mL, and 200 ng/mL, respectively.

The adiponectin chemiluminescence immunoassay kit is capable of performing adiponectin detection using a fully automated chemiluminescence immunoassay analyzer as a detection tool. The experiment shows this adiponectin chemiluminescence immunoassay kit has a detection sensitivity of 0.01 ng/mL, which is at least 10 times more sensitive than a conventional method of detecting adiponectin. This adiponectin chemiluminescence immunoassay kit has a higher detection precision.

When this adiponectin chemiluminescence immunoassay kit is used, the adiponectin calibrator is tested by the fully automated chemiluminescence immunoassay analyzer firstly, and a standard curve is drawn, which is built in computer software. And then an actual sample is tested, and a sample concentration is calculated according to a luminescence value of the sample. Finally, the performances (sensitivity, linearity, precision, and interference) of the adiponectin fully automated chemiluminescence immunoassay system are evaluated.

This adiponectin chemiluminescence immunoassay kit has the following advantages:

1. The acridinium ester is selected as a labeling material and applied in the chemiluminescence immunoassay system. The chemiluminescence system is a direct chemiluminescence, compared with a conventional enzymatic chemiluminescence, the reaction does not require a participation of enzymes, thus saving cost.

2. The chemiluminescence immunoassay system using the acridinium ester or alkaline phosphatase has high detection sensitivity, which can reach 0.01 ng/mL, which is at least 10 times more sensitive than other detection methods.

3. The chemiluminescence immunoassay system using the acridinium ester has a wide linearity range, which can reach more than 200 ng/mL.

4. The acridinium ester chemiluminescence immunoassay system has a high reproducibility, and the intra-assay and inter-assay differences are within 5%, which is difficult to achieve by other chemiluminescence immunoassay systems.

5. The chemiluminescence immunoassay system has achieved the quantification of the sample. By using the built-in standard curve to the test software, the concentration value of the sample can be directly obtained by simply testing the sample.

6. The chemiluminescence immunoassay system can be fully automated, and the addition of reagents and samples is completed by instruments, which makes the operation easier and reduces human error.

In summary, this adiponectin chemiluminescence immunoassay kit has low detection cost, high sensitivity, wide detection linearity range, and high reproducibility, and can be quantitative and simple to operate.

In addition, by using this adiponectin chemiluminescence immunoassay kit, a adiponectin concentration level or distribution of a specific population can be determined. The subject's adiponectin concentration is compared to the adiponectin concentration level of the specific population, if the subject's adiponectin concentration deviates from the adiponectin concentration level of the specific population, the subject is suggested to be unhealthy or weak. Alternatively, continuous adiponectin level monitoring can be performed on the same subject to assess insulin resistance and atherosclerosis, thus predicting the development of type II diabetes and coronary heart diseases, suggesting potential for anti-diabetes, anti-atherosclerosis, and inflammation.

The present disclosure further discloses a method of preparing the aforementioned adiponectin chemiluminescence immunoassay kit, which includes the steps of:

In step S110, an adiponectin monoclonal antibody and a solid-phase carrier are mixed and reacted fully to obtain a solid-phase carrier coated with the adiponectin monoclonal antibody.

In the solid-phase carrier coated with the adiponectin monoclonal antibody, the adiponectin monoclonal antibody can be a human source, genetically engineered or animal source.

In an embodiment, in the solid-phase carrier coated with the adiponectin monoclonal antibody, the solid-phase carrier is a magnetic particle having a linking group for a protein chemical reaction on a surface thereof, a silica-based material having a surface for physical adsorption of proteins, or a magnetic particle subjected to polymer surface treatment.

When the solid-phase carrier is the magnetic particle having the linking group for the protein chemical reaction on the surface thereof or the magnetic particle subjected to polymer surface treatment, preferably, a mass ratio of the adiponectin monoclonal antibody to the magnetic particle ranges from 1:25˜35.

The magnetic particle having the linking group for the protein chemical reaction on the surface thereof can be a magnetic particle having a group selected from the group consisting of: an amino group, a carboxyl group, a tosyl group, and an oxiranyl group on the surface thereof.

The magnetic particle having the linking group for the protein chemical reaction on the surface thereof has a particle size of 0.05 μm to 3 μm.

When the solid-phase carrier is the magnetic particle having the linking group for the protein chemical reaction on the surface thereof in the solid-phase carrier coated with the adiponectin monoclonal antibody, the step S110 is: taking a suspension of the magnetic particle having the linking group for the protein chemical reaction on the surface thereof, resuspending with a buffer followed by magnetic separation to remove supernatant, and then activating a surface linking group on the magnetic particle having the linking group for the protein chemical reaction on the surface thereof by an activator, and then adding the adiponectin monoclonal antibody and reacting fully at room temperature, and removing supernatant by magnetic separation and then resuspending to obtain the magnetic particle having the linking group for the protein chemical reaction on the surface thereof coated with the adiponectin monoclonal antibody.

Taking the solid-phase carrier as the magnetic particle having a carboxyl group on the surface thereof as an example, a preparation process of the solid-phase carrier coated with the adiponectin monoclonal antibody can be as follows: taking a suspension of carboxylated magnetic particles, resuspending with a MES (2-(N-morpholine)ethanesulfonic acid) buffer followed by magnetic separation to remove supernatant, and then adding an EDC (1-ethyl-3-(3-dimethylaminopropyl)-carbodiimide) aqueous solution to activate the surface carboxyl group of the carboxylated magnetic particles, and then adding the adiponectin monoclonal antibody and resuspending at room temperature for 2 h to 10 h, and removing supernatant by magnetic separation and then resuspending with a Tris buffer to obtain the carboxylated magnetic particles coated with the adiponectin monoclonal antibody. MES (2-(N-morpholine)ethanesulfonic acid) buffer has a concentration of 0.02 M and a pH of 5.5. Tris buffer has a concentration of 0.1 M and contains 2% of BSA, and has a pH of 8.0. EDC (1-ethyl-3-(3-dimethylaminopropyl)-carbodiimide) aqueous solution has a concentration of 10 mg/mL to 20 mg/mL, and a ratio of EDC to the carboxylated magnetic particles ranges from 0.05:0.1˜1.

In another embodiment, in the solid-phase carrier coated with the adiponectin monoclonal antibody, the solid-phase carrier is coupled with streptavidin, avidin or neutravidin, and the adiponectin monoclonal antibody is coupled with biotin.

The solid-phase carrier coated with the adiponectin monoclonal antibody may be a commercially available avidin solid-phase carrier, or may be an avidin solid-phase carrier prepared by avidin chemical bond and coupling to other solid-phase carriers.

When the solid-phase carrier is coupled with streptavidin, avidin or neutravidin, and the adiponectin monoclonal antibody is coupled with biotin in the solid-phase carrier coated with the adiponectin monoclonal antibody, the step S110 is: mixing the solid-phase carrier coupled with streptavidin, avidin or neutravidin and the adiponectin monoclonal antibody coupled with biotin in a buffer, and reacting fully to obtain the solid-phase carrier coated with the adiponectin monoclonal antibody.

In step S120, a chemiluminescence marker and a adiponectin monoclonal antibody are mixed and reacted fully to obtain an adiponectin monoclonal antibody labeled with the chemiluminescence marker.

The order of S110 and S120 can be replaced without affecting the method.

In the adiponectin monoclonal antibody labeled with the chemiluminescence marker, the adiponectin monoclonal antibody can be the human source, genetically engineered or animal source.

The adiponectin monoclonal antibodies used in the solid-phase carrier coated with the adiponectin monoclonal antibody and the adiponectin monoclonal antibody labeled with the chemiluminescence marker may be the same or different.

In the adiponectin monoclonal antibody labeled with the chemiluminescence marker, the chemiluminescence marker is isluminol, terpyridine ruthenium, acridinium ester, alkaline phosphatase, or horseradish peroxidase.

Preferably, in a chemiluminescence marker labeled with a statin monoclonal antibody, a ratio of the adiponectin monoclonal antibody to the chemiluminescence marker ranges from 50:1˜10.

In an embodiment, in the adiponectin monoclonal antibody labeled with the chemiluminescence marker, the chemiluminescence marker is coupled with streptavidin, avidin or neutravidin, and the adiponectin monoclonal antibody is coupled with biotin.

When the chemiluminescence marker is coupled with streptavidin, avidin or neutravidin, and the adiponectin monoclonal antibody is coupled with biotin in the adiponectin monoclonal antibody labeled with the chemiluminescence marker, the step S120 is: mixing the chemiluminescence marker coupled with streptavidin, avidin or neutravidin and the adiponectin monoclonal antibody coupled with biotin in a buffer, and reacting fully to obtain the adiponectin monoclonal antibody labeled with the chemiluminescence marker.

Specifically, taking the chemiluminescence marker coupled with streptavidin and the adiponectin monoclonal antibody coupled with biotin as an example, and taking the chemiluminescence marker as acridinium ester as an example, a preparation process of the adiponectin monoclonal antibody labeled with the chemiluminescence marker is as follows: the adiponectin monoclonal antibody solution is taken, 500 μL of phosphate buffer with a pH of 8.0 is added thereto, and 0.1 mg of biotin succinimide (Biotin-NHS) is added and mixed, and is reacted at room temperature in the dark. After 1 h to 2 h, the reaction mixture is taken out and desalted by a centrifugal desalting column. During the desalting process, the treatment is performed firstly by purified water and TBS buffer, respectively, and the liquid in the desalting column is collected and stored to obtain the adiponectin monoclonal antibody solution labeled with the biotin. The streptavidin solution is taken, 0.1 M to 0.2 M of carbonate buffer with a pH of 9.0 to 9.5 is taken and mixed, and then the acridinium ester is added and mixed, and reacted at room temperature in the dark. After 1 h to 2 h, the reaction mixture is taken out and desalted by zeba centrifugal desalting column. During the desalting process, the treatment is performed firstly by purified water and TBS buffer, respectively, and finally the obtained streptavidin labeled with the acridinium ester is added. The adiponectin monoclonal antibody solution labeled with the biotin and the streptavidin labeled with the acridinium ester are mixed to obtain the adiponectin monoclonal antibody labeled with the acridinium ester.

In another embodiment, in the adiponectin monoclonal antibody labeled with the chemiluminescence marker, the chemiluminescence marker is directly linked to the adiponectin monoclonal antibody by a chemical reaction bond or is linked to the adiponectin monoclonal antibody by a protein crosslinking agent.

The protein crosslinking agent can be at least one of a carbonyldiimide salt and a succinimide.

Preferably, the protein crosslinking agent is at least one selected from the group consisting of 1-ethyl-(3-dimethylaminopropyl)carbonyldiimide, 1-ethyl-(3-dimethylaminopropyl)carbonyldiimide hydrochloride, N-hydroxysuccinimide, and sulfonated N-hydroxysuccinimide.

When the chemiluminescence marker is directly linked to the adiponectin monoclonal antibody by a chemical reaction bond in the adiponectin monoclonal antibody labeled with the chemiluminescence marker, the step S120 is: mixing the adiponectin monoclonal antibody and the chemiluminescence marker in a buffer, and reacting fully to obtain the adiponectin monoclonal antibody labeled with the chemiluminescence marker.

Specifically, taking the chemiluminescence marker as the acridinium ester as an example, a process in which the chemiluminescence marker is directly linked to the adiponectin monoclonal antibody by the chemical reaction bond to produce the adiponectin monoclonal antibody labeled with the chemiluminescence marker is as follows: the adiponectin monoclonal antibody solution is taken, a carbonate buffer is added and mixed, and then the acridinium ester is added and mixed, and reacted at room temperature in the dark. After 1 h to 2 h, the reaction mixture is taken out and desalted by the centrifugal desalting column. During the desalting process, the treatment is performed firstly by purified water and TBS buffer, respectively to obtain the adiponectin monoclonal antibody labeled with the acridinium ester.

When the chemiluminescence marker is linked to the adiponectin monoclonal antibody by a protein crosslinking agent in the adiponectin monoclonal antibody labeled with the chemiluminescence marker, the step S120 is: mixing the adiponectin monoclonal antibody, the chemiluminescence marker, and the protein crosslinking agent in a buffer, and reacting fully to obtain the adiponectin monoclonal antibody labeled with the chemiluminescence marker.

Specifically, taking the chemiluminescence marker as alkaline phosphatase and the protein crosslinking agent as 1-ethyl-(3-dimethylaminopropyl)carbonyldiimide hydrochloride and N-hydroxysuccinimide as an example, a process in which the chemiluminescence marker is linked to the adiponectin monoclonal antibody by the protein crosslinking agent to produce the adiponectin monoclonal antibody labeled with the chemiluminescence marker is as follows: 1 mg of adiponectin monoclonal antibody solution and 1 mg of alkaline phosphatase are taken, 500 μL of 2-(N-morpholine)ethanesulfonic acid (MES) buffer with a pH of 5.5 is added, and 1 mg of 1-ethyl-(3-dimethylaminopropyl)carbodiimide hydrochloride and 1 mg of N-hydroxysuccinimide are added and mixed, and reacted at room temperature in the dark. After 1 h to 2 h, the reaction mixture is taken out and desalted by the centrifugal desalting column. During the desalting process, the treatment is performed firstly by purified water and TBS buffer, respectively, and the liquid in the desalting column is collected and stored to obtain the adiponectin monoclonal antibody solution labeled with the alkaline phosphatase.

In alternative embodiments, the adiponectin chemiluminescence immunoassay kit further includes a chemiluminescence substrate solution.

The chemiluminescence substrate solution includes solution A and solution B. The solution A can be a H₂O₂ solution, and the solution B can be a NaOH solution.

In the present embodiment, the solution A is a H₂O₂ solution with a concentration of 0.1 mol/L, and the solution B is a NaOH solution with a concentration of 0.25 mol/L.

In alternative embodiments, the adiponectin chemiluminescence immunoassay kit further includes an adiponectin calibrator.

A preparation process of the adiponectin calibrator is as follows: adiponectin is formulated into a solution of adiponectin at a concentration of 0 to 30 ng/mL using a calibrator buffer.

Specifically, the adiponectin calibrator is a solution of adiponectin at concentrations of 0 ng/mL, 0.5 ng/mL, 10 ng/mL, 20 ng/mL, 80 ng/mL, and 200 ng/mL, respectively.

The present disclosure further discloses a method of detecting a concentration of adiponectin by using the aforementioned adiponectin chemiluminescence immunoassay kit, which includes the steps of:

In step S210, a sandwich immunoassay is performed on a sample to be tested using a solid-phase carrier coated with an adiponectin monoclonal antibody and an adiponectin monoclonal antibody labeled with a chemiluminescence marker. Adiponectin in the sample to be tested reacts with the solid-phase carrier coated with the adiponectin monoclonal antibody and the adiponectin monoclonal antibody labeled with the chemiluminescence marker to form a sandwich complex. A luminescence signal of the sample to be tested is detected after washing and separating.

In step S220, a sandwich immunoassay is performed on an adiponectin calibrator using the solid-phase carrier coated with the adiponectin monoclonal antibody and the adiponectin monoclonal antibody labeled with the chemiluminescence marker. Adiponectin in the adiponectin calibrator reacts with the solid-phase carrier coated with the adiponectin monoclonal antibody and the adiponectin monoclonal antibody labeled with the chemiluminescence marker to form a sandwich complex. A luminescence intensity of the adiponectin calibrator is detected after washing and separating. A standard curve of adiponectin is constructed according to the luminescence intensity of the adiponectin calibrator and the concentration of the adiponectin calibrator, and the standard curve has a horizontal coordinate of concentration and vertical coordinate of luminescence intensity.

The order of S210 and S220 can be replaced without affecting the method.

A preparation process of the adiponectin calibrator is as follows: adiponectin is formulated into a solution of adiponectin at a concentration of 0 to 30 ng/mL using a calibrator buffer.

Specifically, the adiponectin calibrator is a solution of adiponectin at concentrations of 0 ng/mL, 0.5 ng/mL, 10 ng/mL, 20 ng/mL, 80 ng/mL, and 200 ng/mL, respectively.

In step S230, the luminescence signal of the sample to be tested obtained in the step S210 is brought into the standard curve of the adiponectin, and a concentration of the adiponectin in the sample to be tested is calculated.

The following are specific examples.

### Example 1: Preparation of an Adiponectin Chemiluminescence Immunoassay Kit

(1) Preparation of magnetic particles coated with adiponectin monoclonal antibody:

50 mg of a suspension of carboxylated magnetic particles (MagnaBind™, Cat. No. 21353) with a particle size of 0.05 μm to 3 μm was taken, and magnetically separated to remove supernatant, and resuspended with 0.02 M of MES buffer with a pH of 5.5. 0.5 mL to 2 mL of newly formulated 10 mg/mL EDC aqueous solution was added to activate the carboxyl group on the surface of the magnetic beads. 3 mg to 5 mg of adiponectin monoclonal antibody (Novus, NB100-65810) was added and suspended at room temperature for 2 h to 10 h, and magnetically separated to remove supernatant. 0.1 M of Tris buffer containing 2% of BSA with a pH of 8.0 was used to resuspend to 1 mg/mL, thereby obtaining the magnetic particle coated with the adiponectin monoclonal antibody, which were dispensed and stored in a 5 mL portions of each bottle at a temperature of 4° C. for use.

(2) Preparation of adiponectin monoclonal antibody labeled with acridinium ester:

500 μL of 1.0 mg/mL adiponectin monoclonal labeled antibody (Thermo Fisher Scientific, PA1-054) solution was taken, 500 μL of 0.1˜0.2 M carbonate buffer with a pH of 9.0 to 9.5 was added and mixed, and then 10˜20 μL of 5 mg/mL acridinium ester was added and mixed, and reacted at room temperature in the dark. After 1 h to 2 h, the reaction mixture was taken out and desalted by 2 mL of zeba centrifugal desalting column. During the desalting process, the treatment was performed firstly by purified water and TBS buffer, respectively, and finally the obtained solution of the adiponectin monoclonal antibody labeled with the acridinium ester was added. The liquid in the centrifuge tube was collected to a preservation tube to obtain the adiponectin monoclonal antibody labeled with the acridinium ester, which was dispensed and stored in a 1 mL of each bottle at a temperature of 4° C. for use.

(3) Preparation of adiponectin calibrator:

The human sourced adiponectin protein was formulated to concentrations of 0 ng/mL, 0.5 ng/mL, 10 ng/mL, 20 ng/mL, 80 ng/mL, and 200 ng/mL using standard buffer (40 mM Tris-HCl, 0.5% BSA, 1% NaCl, pH 8.0), which was dispensed in 0.5 mL per bottle, and was stored at a temperature of 4° C. for use after lyophilization.

### Example 2: Method for Adiponectin Acridinium Ester Chemiluminescence Immunoassay

The present disclosure used a fully automated chemiluminescence immunoassay analyzer and the adiponectin chemiluminescence immunoassay kit prepared in Example 1 as a detection tool. The methodological mode of the present disclosure was a sandwich method, that is, 50 μL of sample, 50 μL of magnetic particles coated with the adiponectin monoclonal antibody, and 50 μL of adiponectin monoclonal antibody labeled with the acridinium ester were sequentially added by the instrument, and magnetic separation was performed after reacted for 10 min. The instrument fed the reaction mixture into a darkroom, and sequentially added a luminescence substrate solution A (containing 0.1 M of HNO₃ and 0.5% of H₂O₂) and solution B (containing 0.25 M of NaOH) to perform the luminescence reaction. Finally, the luminescence intensity was recorded, and the adiponectin content of the sample to be tested was calculated from the standard curve.

### Example 3: Performance Evaluation of the Adiponectin Chemiluminescence Immunoassay Kit Prepared in Example 1

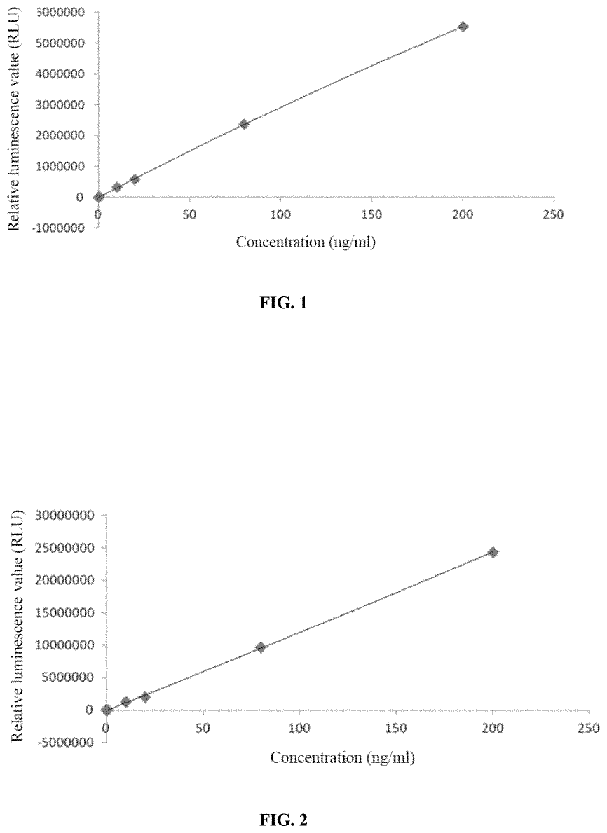

The adiponectin calibrator was tested by using the method in Example 2, and a standard curve was drawn as shown in FIG. 1.

Sensitivity Test:

The sensitivity of the adiponectin chemiluminescence immunoassay kit was calculated with reference to the experimental protocol recommended in the CLSI EP17-A document, and the obtained sensitivity was 0.01 ng/mL.

Linearity Test:

Linear analysis was performed on the standards of concentrations of 0.01 ng/mL, 0.5 ng/mL, 50 ng/mL, 100 ng/mL, and 200 ng/mL, and the linear correlation coefficient was calculated, r=0.9994. In addition, the kit has a linear range of 0.01-200 ng/mL for detection of adiponectin samples.

Precision Measurement:

Two adiponectin samples at concentrations of 0.1 ng/mL and 100 ng/mL were taken. Each of the samples was performed for 3 parallels for each concentration and the detection was performed with three batches of kits. The intra-assay and inter-assay differences of the kit were calculated, and the results showed that the intra-assay and inter-assay differences of that kit were both less than 5%.

Interference Experiment:

The pooled serum was taken, and interfering substances including: conjugated bilirubin, free bilirubin, hemoglobin, ascorbic acid, and glyceride are added, respectively. The adding mass ratio is in accordance with 1:20. The measured values of the pooled serum and the pooled serum after the addition of various interfering substances were measured, respectively, and the deviation between the two was calculated, and the range of ±10% was taken as an acceptable range. The results showed that the interference had reached the NCCLS document standard and can be used for accurate assessment of adiponectin status in clinical laboratories.

Example 4: Comparative Experiment of Adiponectin Chemiluminescence Immunoassay Kit

The adiponectin samples at concentrations of 0 and 0.05 ng/mL were tested by the chemiluminescence detection method in Example 2 and the conventional enzyme linked immunosorbent assay, respectively. The sample at the concentration of 0 ng/mL was subjected to 20 repeated tests to calculate the mean (M) and SD of the sample at the concentration of 0 ng/mL, and the RLU value of M+2SD was brought into the calibration equation to obtain the corresponding concentration value, i.e., the detection sensitivity. The detection sensitivities of the two methods were compared, and the results were shown in the following table:

| | | Chemi- | Enzyme |
|---|---|---|---|
| | | luminescence | linked immuno- |
| Number of | | detection | sorbent assay |
| tests | | (RLU) | (OD) |
| 1 | | 1057 | 0.066 |
| 2 | | 1206 | 0.079 |
| 3 | | 1097 | 0.069 |
| 4 | | 1063 | 0.081 |
| 5 | | 1110 | 0.073 |
| 6 | | 1017 | 0.069 |
| 7 | | 1047 | 0.056 |
| 8 | | 1107 | 0.074 |
| 9 | | 989 | 0.063 |
| 10 | | 1039 | 0.059 |
| 11 | | 1102 | 0.087 |
| 12 | | 1053 | 0.068 |
| 13 | | 1004 | 0.074 |
| 14 | | 1058 | 0.068 |
| 15 | | 1110 | 0.071 |
| 16 | | 1055 | 0.072 |
| 17 | | 1048 | 0.067 |
| 18 | | 1102 | 0.081 |
| 19 | | 992 | 0.067 |
| 20 | | 1080 | 0.069 |
| Mean | | 1067 | 0.071 |
| SD | | 50 | 0.007 |
| M + 2SD | | 1168 | 0.086 |
| 50 pg/mL | 1 | 11206 | 0.133 |
| | 2 | 10936 | 0.141 |
| | 3 | 10498 | 0.142 |
| | Mean | 10880 | 0.139 |

| | | |
|---|---|---|
| Sensitivity (pg/mL) | 0.51 | 10.42 |

As can be seen from the above table, the sensitivity of the chemiluminescence detection method is about 50 times higher than that of the enzyme linked immunosorbent assay.

Example 5: Preparation of an Adiponectin Chemiluminescence Immunoassay Kit

(1) Preparation of magnetic particles coated with adiponectin monoclonal antibody:

50 mg of a suspension of tosylated magnetic particles (Dynal, 30110D) with a particle size of 0.05 μm to 2 μm was taken, and magnetically separated to remove supernatant, and resuspended with borate buffer with a pH of 9.0 to 11.0. 2 mg to 4 mg of adiponectin monoclonal antibody (Novus, NB100-65810) was added, and 0.5 mL to 2 mL of saturated ammonium sulfate solution was added, rotated and mixed, and reacted at a temperature of 37° C. for 20 h to 30 h, and magnetically separated to remove supernatant. 0.1 M of Tris buffer containing 2% of BSA with a pH of 8.0 was used to resuspend to 1 mg/mL, thereby obtaining the magnetic particle coated with the adiponectin monoclonal antibody, which were dispensed and stored in a 5 mL portions of each bottle at a temperature of 4° C. for use.

(2) Preparation of adiponectin monoclonal antibody labeled with alkaline phosphatase:

500 μL of 2.0 mg/mL adiponectin monoclonal labeled antibody (Thermo Fisher Scientific, PA1-054) was taken, and 500 μL of MES acidic buffer with a pH of 5.0 was added, and 1 mg of alkaline phosphatase was added and mixed. Then, 1 mg of 1-ethyl-(3-dimethylaminopropyl)carbodiimide hydrochloride (EDC.HCL) crosslinking agent was added or 1 mg of 1-ethyl-(3-dimethylaminopropyl)carbodiimide hydrochloride (EDC.HCL) and 1 mg of N-hydroxysuccinimide (NHS) mixed crosslinking agents were added, and reacted at room temperature in the dark. After 1 h to 2 h, the reaction mixture is taken out and desalted by the centrifugal desalting column. During the desalting process, the treatment is performed firstly by purified water and TBS buffer, respectively, and the liquid in the desalting tube is collected and stored. The AKTA protein on the desalting solution was purified by a purification system, and the adiponectin monoclonal antibody labeled with alkaline phosphatase was collected and obtained, which was dispensed and stored in a 1 mL of each bottle at a temperature of 4° C. for use.

(3) Preparation of adiponectin calibrator:

The human sourced adiponectin protein was formulated to concentrations of 0 ng/mL, 0.5 ng/mL, 10 ng/mL, 20 ng/mL, 80 ng/mL, and 200 ng/mL using standard buffer (40 mM Tris-HCl, 0.5% BSA, 1% NaCl, pH 8.0), which was dispensed in 0.5 mL per bottle, and was stored at a temperature of 4° C. for use after lyophilization.

Example 6: Method for Adiponectin Alkaline Phosphatase Chemiluminescence Immunoassay

The present disclosure used a fully automated chemiluminescence immunoassay analyzer and the adiponectin chemiluminescence immunoassay kit prepared in Example 5 as a detection tool. The methodological mode of the present disclosure was a sandwich method, that is, 20 μL of sample, 50 μL of magnetic particles coated with the adiponectin monoclonal antibody, and 50 μL of adiponectin antibody labeled with the alkaline phosphatase were sequentially added by the instrument, and magnetic separation was performed after reacted for 10 min. The instrument fed the reaction mixture into a darkroom, and added AMPPD luminescence substrate to perform the luminescence reaction. Finally, the luminescence intensity was recorded, and the adiponectin content of the sample to be tested was calculated from the standard curve.

Example 7: Performance Evaluation of the Adiponectin Chemiluminescence Immunoassay Kit Prepared in Example 5

The adiponectin calibrator was tested by using the method in Example 6, and a standard curve was drawn as shown in FIG. 2.

Sensitivity Test:

The sensitivity of the adiponectin chemiluminescence immunoassay kit was calculated with reference to the experimental protocol recommended in the CLSI EP17-A document, and the obtained sensitivity was 0.02 ng/mL.

Example 8: Preparation of an Adiponectin Chemiluminescence Immunoassay Kit

(1) Preparation of magnetic particles coated with adiponectin monoclonal antibody.

It is consistent with (1) in Example 5.

(2) Preparation of adiponectin monoclonal antibody labeled with biotin:

500 μL of 2.0 mg/mL adiponectin monoclonal labeled antibody was taken, and 500 μL of phosphate buffer with a pH of 8.0 was added, and 0.1 mg of biotin succinimide (Biotin-NHS) was added and mixed, and reacted at room temperature in the dark. After 1 h to 2 h, the reaction mixture was taken out and desalted by a centrifugal desalting column. During the desalting process, the treatment was performed firstly by purified water and TBS buffer, respectively, and the liquid in the desalting tube was collected and stored. The adiponectin monoclonal antibody solution labeled with the biotin was obtained, which was dispensed and stored in a 1 mL of each bottle at a temperature of 4° C. for use.

(3) Preparation of streptavidin labeled with acridinium ester:

500 μL of 1.0 mg/mL streptavidin was taken, 500 μL of 0.1-0.2 M carbonate buffer with a pH of 9.0-9.5 was added and mixed, and then 10-20 μL of 5 mg/mL acridinium ester was added and mixed, and reacted at room temperature in the dark. After 1 h to 2 h, the reaction mixture was taken out and desalted by 2 mL of zeba centrifugal desalting column. During the desalting process, the treatment was performed firstly by purified water and TBS buffer, respectively, and finally the obtained streptavidin labeled with the acridinium ester was added. The liquid in the centrifuge tube was collected to a preservation tube to obtain the streptavidin labeled with the acridinium ester, which was dispensed and stored in a 1 mL of each bottle at a temperature of 4° C. for use.

(4) Preparation of adiponectin monoclonal antibody labeled with acridinium ester: the adiponectin monoclonal antibody solution labeled with the biotin and the streptavidin labeled with the acridinium ester were mixed at a ratio of 4:1 to obtain the adiponectin monoclonal antibody labeled with the acridinium ester.

(5) Preparation of adiponectin calibrator:

The human sourced adiponectin protein was formulated to concentrations of 0 ng/mL, 0.5 ng/mL, 10 ng/mL, 20 ng/mL, 80 ng/mL, and 200 ng/mL using standard buffer (40 mM Tris-HCl, 0.5% BSA, 1% NaCl, pH 8.0), which was dispensed in 0.5 mL per bottle, and was stored at a temperature of 4° C. for use after lyophilization.

Example 9: Method for Immunoassay of Adiponectin by Avidin-Biotin System

A fully automated chemiluminescence immunoassay analyzer and the adiponectin chemiluminescence immunoassay kit prepared in Example 8 were used as a detection tool. In other words, 10 μL of sample, 50 μL of magnetic particles coated with the adiponectin monoclonal antibody, 50 μL of the adiponectin antibody labeled with the biotin, and 50 μL of the streptavidin labeled with the acridinium ester were sequentially added by the instrument, and magnetic separation was performed after reacted for 20 min. The instrument fed the reaction mixture into a darkroom, and sequentially added a luminescence substrate solution A (containing 0.1 M of HNO₃ and 0.5% of H₂O₂) and solution B (containing 0.25 M of NaOH) to perform the luminescence reaction. Finally, the luminescence intensity was recorded, and the adiponectin content of the sample to be tested was calculated from the standard curve.

Example 10: Performance Evaluation of the Adiponectin Chemiluminescence Immunoassay Kit Prepared in Example 8

Sensitivity Test:

The sensitivity of the adiponectin chemiluminescence immunoassay kit was calculated with reference to the experimental protocol recommended in the CLSI EP17-A document, and the obtained sensitivity was 0.005 ng/mL.

The foregoing descriptions are merely specific embodiments of the present invention, but are not intended to limit the protection scope of the present invention. Any variation or replacement readily figured out by a person skilled in the art within the technical scope disclosed in the present invention shall all fall within the protection scope of the present invention. Therefore, the protection scope of the present invention shall be subject to the protection scope of the appended claims.

1. An adiponectin chemiluminescence immunoassay kit, comprising:
a solid-phase carrier coated with an adiponectin monoclonal antibody; and
an adiponectin monoclonal antibody labeled with a chemiluminescence marker.

2. The adiponectin chemiluminescence immunoassay kit according to claim 1, wherein in the solid-phase carrier coated with the adiponectin monoclonal antibody, the solid-phase carrier is a magnetic particle having a linking group for a protein chemical reaction on a surface thereof.

3. The adiponectin chemiluminescence immunoassay kit according to claim 2, wherein the magnetic particle having the linking group for the protein chemical reaction on the surface thereof is a magnetic particle having a group selected from the group consisting of: an amino group, a carboxyl group, a tosyl group, and an oxiranyl group on the surface thereof.

4. The adiponectin chemiluminescence immunoassay kit according to claim 2, wherein the magnetic particle having the linking group for the protein chemical reaction on the surface thereof has a particle size of 0.05 μm to 3 μm.

5. The adiponectin chemiluminescence immunoassay kit according to claim 1, wherein in the solid-phase carrier coated with the adiponectin monoclonal antibody, the solid-phase carrier is coupled with streptavidin, avidin or neutravidin; and the adiponectin monoclonal antibody is coupled with biotin.

6. The adiponectin chemiluminescence immunoassay kit according to claim 1, wherein in the adiponectin monoclonal antibody labeled with the chemiluminescence marker, the chemiluminescence marker is isluminol, terpyridine ruthenium, acridinium ester, alkaline phosphatase, or horseradish peroxidase.

7. The adiponectin chemiluminescence immunoassay kit according to claim 1, wherein in the adiponectin monoclonal antibody labeled with the chemiluminescence marker, the chemiluminescence marker is coupled with streptavidin, avidin or neutravidin, and the adiponectin monoclonal antibody is coupled with biotin.

8. The adiponectin chemiluminescence immunoassay kit according to claim 1, wherein in the adiponectin monoclonal antibody labeled with the chemiluminescence marker, the chemiluminescence marker is directly linked to the adiponectin monoclonal antibody by a chemical reaction bond or is linked to the adiponectin monoclonal antibody by a protein crosslinking agent.

9. The adiponectin chemiluminescence immunoassay kit according to claim 8, wherein the protein crosslinking agent is at least one selected from the group consisting of a carbonyldiimide salt and a succinimide.

10. The adiponectin chemiluminescence immunoassay kit according to claim 9, wherein the protein crosslinking agent is at least one selected from the group consisting of 1-ethyl-(3-dimethylaminopropyl)carbonyldiimide, 1-ethyl-(3-dimethylaminopropyl)carbonyldiimide hydrochloride, N-hydroxysuccinimide, and sulfonated N-hydroxysuccinimide.

11. The adiponectin chemiluminescence immunoassay kit according to claim 1, further comprising an adiponectin calibrator.

12. A method of preparing an adiponectin chemiluminescence immunoassay kit of claim 1, comprising:
mixing an adiponectin monoclonal antibody and a solid-phase carrier and fully reacting to obtain a solid-phase carrier coated with the adiponectin monoclonal antibody; and mixing a chemiluminescence marker and a adiponectin monoclonal antibody and reacting fully to obtain an adiponectin monoclonal antibody labeled with the chemiluminescence marker.

13. The method according to claim 12, wherein in the solid-phase carrier coated with the adiponectin monoclonal antibody, the solid-phase carrier is a magnetic particle having a linking group for a protein chemical reaction on a surface thereof;
the operation of mixing the adiponectin monoclonal antibody and the solid-phase carrier and reacting fully to obtain the solid-phase carrier coated with the adiponectin monoclonal antibody is: taking a suspension of the magnetic particle having the linking group for the protein chemical reaction on the surface thereof, resuspending with a buffer followed by magnetic separation to remove supernatant, and then activating a surface linking group on the magnetic particle having the linking group for the protein chemical reaction on the surface thereof by an activator, and then adding the adiponectin monoclonal antibody and reacting fully at room temperature, and removing supernatant by magnetic separation and then resuspending to obtain the magnetic particle having the linking group for the protein chemical reaction on the surface thereof coated with the adiponectin monoclonal antibody.

14. The method according to claim 12, wherein in the solid-phase carrier coated with the adiponectin monoclonal antibody, the solid-phase carrier is coupled with streptavidin, avidin or neutravidin, and the adiponectin monoclonal antibody is coupled with biotin;
the operation of mixing the adiponectin monoclonal antibody and the solid-phase carrier and reacting fully to obtain the solid-phase carrier coated with the adiponectin monoclonal antibody is: mixing the solid-phase carrier coupled with streptavidin, avidin or neutravidin and the adiponectin monoclonal antibody coupled with biotin in a buffer, and reacting fully to obtain the solid-phase carrier coated with the adiponectin monoclonal antibody.

15. The method according to claim 12, wherein in the adiponectin monoclonal antibody labeled with the chemiluminescence marker, the chemiluminescence marker is coupled with streptavidin, avidin or neutravidin, and the adiponectin monoclonal antibody is coupled with biotin;
the operation of mixing the chemiluminescence marker and the adiponectin monoclonal antibody and reacting fully to obtain the adiponectin monoclonal antibody labeled with the chemiluminescence marker is: mixing the chemiluminescence marker coupled with streptavidin, avidin or neutravidin and the adiponectin monoclonal antibody coupled with biotin in a buffer, and reacting fully to obtain the adiponectin monoclonal antibody labeled with the chemiluminescence marker.

16. The method according to claim 12, wherein in the adiponectin monoclonal antibody labeled with the chemiluminescence marker, the chemiluminescence marker is directly linked to the adiponectin monoclonal antibody by a chemical reaction bond;
the operation of mixing the chemiluminescence marker and the adiponectin monoclonal antibody and reacting fully to obtain the adiponectin monoclonal antibody labeled with the chemiluminescence marker is: mixing the adiponectin monoclonal antibody and the chemiluminescence marker in a buffer, and reacting fully to obtain the adiponectin monoclonal antibody labeled with the chemiluminescence marker.

17. The method according to claim 12, wherein in the adiponectin monoclonal antibody labeled with the chemiluminescence marker, the chemiluminescence marker is linked to the adiponectin monoclonal antibody by a protein crosslinking agent;
the operation of mixing the chemiluminescence marker and the adiponectin monoclonal antibody and reacting fully to obtain the adiponectin monoclonal antibody labeled with the chemiluminescence marker is: mixing the adiponectin monoclonal antibody, the chemiluminescence marker, and the protein crosslinking agent in a buffer, and reacting fully to obtain the adiponectin monoclonal antibody labeled with the chemiluminescence marker.

18. A method of detecting a concentration of adiponectin, by using an adiponectin chemiluminescence immunoassay kit of claim 1, wherein the method of detecting the concentration of the adiponectin comprises the steps of:
performing a sandwich immunoassay on a sample to be tested using a solid-phase carrier coated with an adiponectin monoclonal antibody and an adiponectin monoclonal antibody labeled with a chemiluminescence marker; wherein adiponectin in the sample to be tested reacts with the solid-phase carrier coated with the adiponectin monoclonal antibody and the adiponectin monoclonal antibody labeled with the chemiluminescence marker to form a sandwich complex; and a luminescence intensity of the sample to be tested is detected after washing and separating;
performing a sandwich immunoassay on an adiponectin calibrator using the solid-phase carrier coated with the adiponectin monoclonal antibody and the adiponectin monoclonal antibody labeled with the chemiluminescence marker; wherein adiponectin in the adiponectin calibrator reacts with the solid-phase carrier coated with the adiponectin monoclonal antibody and the adiponectin monoclonal antibody labeled with the chemiluminescence marker to form a sandwich complex; a luminescence intensity of the adiponectin calibrator is detected after washing and separating; and a standard curve of adiponectin is constructed according to the luminescence intensity of the adiponectin calibrator and the concentration of the adiponectin calibrator, the standard curve has a horizontal coordinate of concentration and vertical coordinate of luminescence intensity; and
bringing a luminescence signal of the sample to be tested into the standard curve of the adiponectin, and calculating a concentration of the adiponectin in the sample to be tested.
